(19) United States
(12) Patent Application Publication
(10) Pub. No.: US 2009/0143362 A1
(43) Pub. Date: Jun. 4, 2009

(54) CARBAMAZEPINE FORMULATIONS

(76) Inventors: Umesh Vinayakrao BARABDE, Amravati (IN); Rajan Kumar Verma, New Delhi (IN); Rajeev Singh Raghuvanshi, Gurgaon (IN)

Correspondence Address: Jayadeep R. Deshmukh, Esq.;Ranbaxy Inc., Suite 2100, 600 College Road East,, Princeton, NJ, 08540, US

(21) Appl. No.: 12/328,027

(22) Filed: Dec. 4, 2008

(30) Foreign Application Priority Data
Dec. 4, 2007 (IN) 2543/DEL/2007

(51) Int. Cl. A61K 31/55; A61P 25/08

(52) U.S. Cl. 514/217

(57) ABSTRACT

The present invention relates to a process for preparing a pharmaceutical composition comprising carbamazepine that has a constant release profile.

### FIELD OF THE INVENTION

The present invention relates to a process for preparing a pharmaceutical composition comprising carbamazepine that has constant release profile.

### BACKGROUND OF THE INVENTION

Carbamazepine, 5H-dibenz-[b,f]azepine-5-carboxamide, is a well established anti-epileptic compound. It is regarded as a first-line drug in the treatment of patients suffering from partial seizures, with and without second generalization, and in patients with generalized tonic-clonic seizures. Besides being an antiepileptic compound, carbamazepine has also proved effective in the treatment of pain associated with trigeminal neuralgia and in patients suffering from manic-depressive illness, post therapeutic neuralgia, or phantom limb pain. Additionally, carbamazepine is used in various psychiatric disorders such as bipolar disorder, depression, cocaine addiction, alcohol addiction and other obsessive compulsive disorders and cardiovascular diseases. The drug appears to act by reducing postsynaptic responses and by blocking post-tetanic potentiation.

Carbamazepine is a poorly water-soluble drug. Pharmacokinetic studies have shown it to be slowly and erratically absorbed from the gastrointestinal tract when administered in oral dosage form. Although the half-life of carbamazepine is relatively long, between 25 and 85 hours after a single dose, however, due to autoinduction, its effect is substantially reduced after repeated dosing. Due to its increased metabolism, pronounced daily fluctuations in the serum concentration of carbamazepine are observed and are of concern. The therapeutic range of carbamazepine is about 4-12 μml. Blood levels of carbamazepine below 4 μg/ml have been found ineffective in treating clinical disorders and blood levels greater than 12 μg/ml have been found to be likely to result in side-effects such as neuromuscular disorders, cardiovascular and gastrointestinal effects. Multiple dosing in this case may lead to undesirable fluctuations in the plasma concentration of the active substances.

Another major problem associated with carbamazepine is that it exhibits polymorphism. Crystal characterization has identified three main polymorphs of carbamazepine and a dihydrate. The polymorphs and dihydrate of carbamazepine exhibit different dissolution rates and bioavailabilities. There have been cases of bioinequivalence and clinical failure which may be due to polymorphism. In presence of water, carbamazepine transforms rapidly into carbamazepine dihydrate. Carbamazepine dihydrate crystals grow by the whisker mechanism and conversion has been shown by X-ray powder diffraction to be 95% complete after 1 hour.

The inhibition of formation of large crystals of carbamazepine dihydrate are of great importance for its pharmaceutical formulation since large crystals of carbamazepine dissolve slowly and unpredictably and, therefore, cause bioavailability problems and may result in unpredictable and uncontrollable drug delivery. The avoidance of the formation of these crystals is important especially for carbamazepine since it exhibits a very narrow therapeutic index.

Some attempts to overcome the above problems were made. For example, Khanna S. C., et al., U.S. Pat. No. 4,857,336, have described an oral dosage form for administration of carbamazepine wherein a core comprising a paste of a fine carbamazepine powder dissolved in a protective colloid, a hydrophilic swelling agent and, optionally, a water-soluble osmosis inducing-agent was encapsulated in a water-permeable shell impermeable to the components of the core.

Another attempt is described in U.S. Pat. No. 5,284,662, which described the usage of minimum organic solvents over the U.S. Pat. No. 4,857,336, particularly in core preparations.

U.S. Pat. No. 5,980,942 describes a zero order sustained release matrix tablet formulation of carbamazepine. The matrix tablet formulation comprises a hydrophilic polymer gel that inhibits transformation of carbamazepine into carbamazepine dihydrate and effectively changes the anhydrous carbamazepine into an amorphous form that can be released from the matrix by zero-order release kinetics.

U.S. Pat. Nos. 5,326,570 and 5,912,013, assigned to Shire disclose drug delivery systems consisting of a single dosage form containing three types of units: immediate release unit, sustained release unit and enteric release unit, capable of releasing carbamazepine at varying times.

Although aqueous granulations have been utilized since these have been shown to provide granules exhibiting less conversion, however performing these granulations in a high shear mixer or a fluid bed can cause dihydrate formation and the subsequent drying can cause desolvation. Carbamazepine is transformed from one anhydrate to another through a dihydrate intermediate during these processes.

It is an object of this invention to provide a process for preparing a carbamazepine composition that inhibits the transformation of anhydrous carbamazepine into crystallized dihydrate form and to provide a composition that has a constant release profile.

### SUMMARY OF THE INVENTION

It is one of the aspects to provide a process for preparing an oral pharmaceutical composition comprising carbamazepine that has a constant release profile, which involves granulating with organic solvents.

It is another aspect to provide a process for preparing an oral pharmaceutical composition comprising carbamazepine, wherein the process comprises the steps of:
(i) blending carbamazepine with one or more pharmaceutically acceptable excipients to form a uniform mixture,
(ii) granulating the mixture with one or more organic solvents,
(iii) drying the granulate to obtain discrete units, and
(iv) processing the units into an oral pharmaceutical composition.

Embodiments of the process may include one or more of the following features. For example, granulating the carbamazepine blend with one or more organic solvents may be carried out by addition of required quantity of organic solvent to the mixture/blend which is sufficient to wet the mixture completely or partially.

The granulate obtained may be formulated as plurality of discrete or aggregated units of particles, spheroids, seeds, pellets, beads, granules, mini-tablets or tablets. These may be further processed into immediate-release, controlled-release and/or delayed-release compositions. Several of these units may be compressed using conventional techniques or filled as such in capsules to form immediate release compositions.

Controlled-release and/or delayed-release compositions may be formulated by using one or more controlled-release or enteric polymers. These polymers may be added as matrix-forming polymers or applied as coatings.

In another embodiment, a mixture of immediate-release, controlled-release and delayed-release units may be compressed together into tablets or filled into capsules to provide a continuous release throughout the gastrointestinal tract.

According to one of the embodiments, the composition of the present invention may include a mixture of different release units of carbamazepine to provide a constant release profile up to about 24 hours time period so as to maintain the carbamazepine blood level within the therapeutic range. Particularly, the mixture may include controlled-release and delayed-release units. More particularly the ratio of controlled-release to the delayed-release units may range from about 20:80 to about 80:20 by weight.

The pharmaceutically acceptable excipients may be one or more of diluents, binders, disintegrants, lubricants, glidants, coloring agents and flavoring agents.

It is yet another aspect to provide a method of treating convulsions, trigeminal neuralgia or bipolar disorder, by administering the oral pharmaceutical composition comprising carbamazepine that has a more predictable and constant release profile.

The method may further include administering other anticonvulsant or pharmaceutical agents.

The details of one or more embodiments of the inventions are set forth in the description below. Other features and objects of the invention will be apparent from the description and claims.

### DETAILED DESCRIPTION OF THE INVENTION

The inventors have developed a process for preparing carbamazepine composition that inhibits the transformation of anhydrous carbamazepine into crystallized dihydrate form that helps to achieve constant release profile using simple manufacturing technique. We found that granulating carbamazepine or mixture containing carbamazepine with one or more organic solvents in the process of preparing carbamazepine core significantly reduces the formation of dihydrate crystals.

The oral pharmaceutical composition comprising carbamazepine can be prepared by any of the methods known in the art and also by the processes described in our co-pending Indian Patent application Nos. 1380/DEL/2005, 1382/DEL/2005 and 145/DEL/2006, which are incorporated herein their entirety. The present invention describes the usage of organic solvents in preparing carbamazepine core that provides constant release profile.

The composition of the present invention can be a simple admixture, plurality of discrete or aggregated units of particles, spheroids, seeds, pellets, beads, granules, mini-tablets or tablets.

The granulation method involves granulating carbamazepine and excipients, with one or more organic solvents or dispersion of a binder and preparing carbamazepine core unit of desired size. The core unit may be prepared by the techniques known in the field of art, for example, simple granulation, followed by sieving; granulation followed by tabletization into mini-tablets or tablets; extrusion and marumerization or spheronization; rotogranulation; pelletization; micropelletization, etc. These steps may be carried out in the conventional manner.

The composition may be formulated into immediate release, controlled-release or delayed-release units.

The term “granulate” as used herein includes plurality of discrete or aggregated units in the form of particles, spheroids, seeds, pellets, beads, granules, mini-tablets or tablets and any other matrix systems. The “unit” may also include mixture of different types of units mentioned here.

The immediate-release carbamazepine units can be formulated by the process comprising steps of:
(i) blending carbamazepine with one or more pharmaceutically acceptable excipients to form a uniform mixture,
(ii) granulating the mixture with one or more organic solvents,
(iii) drying the granulate to obtain discrete units, and
(iv) processing the units into an oral pharmaceutical composition.

The controlled-release carbamazepine units can be formulated by providing a coating of controlled-release polymer over the immediate release units. The controlled-release units can also be prepared by blending carbamazepine with controlled-release polymers and other pharmaceutically acceptable excipients to form matrix-type carbamazepine units.

The term “controlled-release”, as used herein, includes any type of controlled-release preparations such as prolonged release, sustained release, modified release and extended release.

The delayed-release units can be prepared by providing a coating of enteric polymers over carbamazepine containing granulate/unit. The unit may be immediate release or controlled-release. The enteric polymers are selected from any such pharmaceutically acceptable enteric polymers, which would facilitate erosion and breakdown of the units at a pH of 4.5 and above.

The controlled-release and delayed-release units may be combined in a desired ratio to provide a constant release profile up to about 24 hours time period so as to maintain the carbamazepine blood level within the therapeutic range. The ratio of controlled-release unit to the delayed-release unit in the composition may range from about 20:80 to about 80:20 by weight. The units may be filled into capsules or sachets or compressed into tablets.

Suitable organic solvents used in preparing granulating dispersion include, but are not limited to alcohols such as C₁ to C₁₂ alcohols, diols, triols or aromatic alcohols; ketones such as acetone or ethyl methyl ketone; halogenated hydrocarbons such dichloro methane or trichloromethane; or mixture thereof. Particularly the alcohols may be methanol, ethanol, propanol, butanol, isopropyl alcohol and the mixtures thereof.

The controlled-release polymer used in preparing controlled-release units can be selected from one or more of pharmaceutically acceptable polymers, which can control the rate of release of carbamazepine, i.e., cellulose derivatives, starch, gums, alginates, polyvinyl pyrrolidone, acrylic acid derivatives and polyethylene oxides.

Suitable examples of cellulosic polymers include, but are not limited to, ethylcellulose, hydroxypropyl methylcellulose, hydroxypropyl cellulose, methylcellulose, carboxymethylcellulose, hydroxymethylcellulose and hydroxyethylcellulose.

Suitable examples of acrylic acid derivatives include, but are not limited to, polymethacrylates such as ethyl acrylate/methyl methacrylate copolymer (Eudragit® NE-30-D) and ammonio methacrylate copolymer types A and B (Eudragit® RL30D and RS30D).

The polyethylene oxide (Polyox®) may be chosen from the ones having average molecular weight between 100,000 and 7,000,000, or a mixture of two or more polyethylene oxides with different molecular weights may be also used.

Suitable enteric polymers include, but are not limited to, cellulose acetate phthalate, cellulose acetate trimellitate, hydroxypropyl methylcellulose acetate phthalate, polyvinyl acetate phthalate, hydroxypropyl methylcellulose phthalate, hydroxypropyl methylcellulose acetate succinate; methacrylic acid copolymers such as Eudragit® L 100-55, D-55, 100, and Eudragit® S 100, and mixtures thereof.

Suitable solvents used for preparing a solution of controlled-release polymer or enteric polymers include, but are not limited to water, alcohols such as ethyl alcohol or isopropyl alcohol; ketones such as acetone or ethylmethyl ketone; halogenated hydrocarbons such dichloro methane or trichloromethane; or mixture thereof.

The coating may be done using a conventional coating pan, a spray coater, a rotating perforated pan, or an automated system, such as a centrifugal fluidizing (CF) granulator, a fluidized bed process, or any other suitably automated coating equipment.

The composition may additionally comprise surfactants and pH-modifiers.

Suitable surfactant can be anionic, cationic, zwitterionic and nonionic surfactants.

Particularly, the compositions include at least one anionic surfactant. Suitable anionic surfactants include but are not limited to alkyl sulfonates, alkyl phosphates, alkyl phosphonates, potassium laurate, sodium lauryl sulfate, sodium dodecylsulfate, alkyl polyoxyethylene sulfates, dioctyl sodium sulfosuccinate, phosphatidyl glycerol, phosphatidylinositol, diphosphatidylglycerol, phosphatidyl inosine, phosphatidylserine, phosphatidic acid and their salts, cholic acid and other bile acids (e.g., cholic acid, deoxycholic acid, glycocholic acid, taurocholic acid, glycodeoxycholic acid) and salts thereof (e.g., sodium deoxycholate, etc.).

Suitable pH-modifiers include, but are not limited to, citric acid, sodium bicarbonate, monosodium citrate, trisodium citrate, tribasic sodium phosphate, sodium chloride or mixtures thereof.

The term “pharmaceutically acceptable excipients”, as used herein, includes one or more of diluents, binders, disintegrants, lubricants, glidants, coloring agents and flavoring agents.

Suitable diluents include, but are not limited to, corn starch, lactose, white sugar, sucrose, sugar compressible, sugar confectioners, glucose, sorbitol, calcium carbonate, calcium phosphate-dibasic, calcium phosphate-tribasic, calcium sulfate, microcrystalline cellulose, silicified microcrystalline cellulose, cellulose powdered, dextrates, dextrins, dextrose, fructose, kaolin, lactitol, mannitol, starch and starch pregelatinized.

Suitable binders include, but are limited to methyl cellulose, hydroxypropyl cellulose [low viscosity (L), medium viscosity (M) or high viscosity (H)], hydroxypropyl methylcellulose, polyvinylpyrrolidone (povidone), copolymer of polyvinylpyrrolidone and vinyl acetate (copovidone), gelatin, gum arabic, ethyl cellulose, polyvinyl alcohol, pullulan, pregelatinized starch, agar, tragacanth, sodium alginate, and propylene glycol.

Suitable lubricants and glidants include, but are not limited to, colloidal anhydrous silica, stearic acid, magnesium stearate, calcium stearate, talc, hydrogenated castor oil, sucrose esters of fatty acids, microcrystalline wax, yellow beeswax and white beeswax.

The controlled-release layer or enteric layer may additionally comprise plasticizers, coloring agents, lubricants, antiadherents, etc.

Suitable plasticizers include, but are not limited to, propylene glycol, triethylene glycol, oleic acid, triethylcitrate, tributylcitrate, triacetin, diethyl phthalate, dibutyl phthalate, dibutylsebacate, glyceryl monostearate, castor oil, ethylene glycol monooleate.

The coloring agents and flavoring agents of the present invention may be selected from any FDA approved colors and flavors for oral use.

The present invention is illustrated below by reference to the following example. However, one skilled in the art will appreciate that the specific methods and results discussed are merely illustrative of the invention, and not to be construed as limiting the invention.

### EXAMPLE 1

### Preparation of Carbamazepine Containing Units

### Unit I:

| S. No. | Ingredients | % w/w |
|---|---|---|
| 1 | Carbamazepine | 76.92 |
| 2 | Microcrystalline cellulose | 2.31 |
| 3 | Lactose monohydrate | 3.18 |
| 4 | Citric acid | 4.81 |
| 5 | Sodium lauryl sulphate | 0.47 |
| 6 | Polyvinylpyrrolidone | 2.56 |
| 7 | Hydoxypropylcellulose | 3.85 |

Granulating dispersion (~18% w/w) in Isopropyl alcohol

| | | |
|---|---|---|
| 8 | Polyvinylpyrrolidone | 3.59 |
| 9 | Talc | 1.73 |
| 10 | Polyethylene glycol-400 | 0.58 |

### Procedure:

1. Carbamazepine, microcrystalline cellulose, lactose, citric acid, sodium lauryl sulfate, hydroxypropylcellulose and a part of polyvinylpyrrolidone were mixed and granulated with granulating dispersion containing talc, remaining part of polyvinylpyrrolidone and polyethylene glycol, which is prepared in isopropyl alcohol.
2. The granulate was dried and sieved to get granules (units) of desired particle size.

### Unit II:

Composition of carbamazepine granules—same as Unit I.

Granulating dispersion (˜18% w/w) prepared in Isopropyl alcohol+Water (1:1) mixture.

### Unit III:

Composition of carbamazepine granules—same as Unit I.

Granulating dispersion (˜18% w/w) prepared in Methanol.

### Unit IV:

Composition of carbamazepine granules—same as Unit I.

Granulating dispersion (˜18% w/w) prepared in Methanol+Water (1:1) mixture.

### Unit V:

Composition of carbamazepine granules—same as Unit I.

Granulating dispersion (˜18% w/w) prepared in Water.

### In-Vitro Dissolution Studies

In-vitro dissolution studies of the above carbamazepine units were conducted in 0.1N HCl using USP-2 dissolution apparatus at 50 rpm. The release profile is shown in Table 1.

TABLE 1

| | % Drug dissolved | | | | |
|---|---|---|---|---|---|
| | | Unit II | | | |
| | | Prepared using | | Unit IV | |
| | Unit I | Isopropyl | | Prepared using | Unit V |
| | Prepared using | alcohol + | Unit III | Methanol + | Prepared |
| | Isopropyl | Water (1:1) | Prepared using | Water (1:1) | using |
| | alcohol as | mixture as | Methanol as | mixture as | Water as |
| | granulating | granulating | granulating | granulating | granulating |
| Time (h) | solvent | solvent | solvent | solvent | solvent |
| 0.00 | 0 | 0 | 0 | 0 | 0 |
| 0.17 | 39 | 10 | 41 | 21 | 36 |
| 0.35 | 60 | 15 | 52 | 37 | 46 |
| 0.50 | 72 | 18 | 62 | 47 | 55 |
| 1 | 87 | 35 | 80 | 63 | 72 |
| 2 | 101 | 43 | 94 | 77 | 89 |

### EXAMPLE 2

### Preparation of Controlled-Release Carbamazepine Composition

| | | Percent |
|---|---|---|
| | Ingredients | w/w |
| Unit Granules | Carbamazepine | 86.00 |
| | containing Unit I of Example 1 | |
| Controlled- | Acrylic and methacrylic acid esters | 6.11 |
| release | copolymer dispersion | |
| Coating | (30% aq. dispersion) | |
| | Polyvinylpyrrolidone | 2.62 |
| | Triethyl citrate | 1.08 |
| | Colloidal silicon dioxide | 0.45 |
| | Talc | 1.79 |
| Enteric | Methacrylic acid and ethyl acrylate | 8.23 |
| Coating | copolymer dispersion | |
| | Polyvinylpyrrolidone | 1.16 |
| | Triethyl citrate | 1.65 |
| | Colloidal silicon dioxide | 0.34 |
| | Talc | 0.67 |
| Seal Coating | Hydroxypropyl methylcellulose | 1.57 |
| | Talc | 0.40 |

### Procedure:

1. The carbamazepine Unit I of Example 1 was divided into two parts.
2. The first part was coated with aqueous dispersion of controlled-release polymer and the second part was coated with aqueous dispersion of enteric polymer.
3. Both the coated units were further coated with seal coating mixture.
4. The two types of units were blended in a 65:35 weight ratio and filled in the capsules.

### In-Vitro Dissolution Studies

A comparative in-vitro dissolution study of the above capsules of Example 2 (equivalent to 300 mg) against Carbatrol®-300 mg (Shire Inc., USA) was conducted in change over media (0.1N HCl for 4 hours and phosphate buffer, pH 6.8 with 0.5% sodium lauryl sulfate for remaining time) using USP-2 dissolution apparatus at 100 rpm. The release profile is shown in Table 2.

TABLE 2

% Drug dissolved

| Time (h) | Carbatrol (300 mg) | Example 2 |
|---|---|---|
| 0 | 0 | 0 |
| 0.5 | 12 | 12 |
| 1 | 23 | 23 |
| 2 | 41 | 37 |
| 4 | 52 | 56 |
| 4.5 | 75 | 80 |
| 5 | 89 | 95 |
| 6 | 102 | 106 |
| 8 | 103 | 110 |
| 12 | 100 | 106 |

While several particular forms of the invention have been illustrated and described, it will be apparent that various modifications and combinations of the invention detailed in the text can be made. For example, the carbamazepine units used for preparing the enteric release unit does not necessarily need to include only immediate release core of carbamazepine but instead can be made up of a controlled-release unit of carbamazepine, e.g., immediate release unit of carbamazepine is first coated with controlled-release polymer and then coated with enteric-release polymer. Also, the enteric-release unit can be made up of mixture of units containing immediate release carbamazepine and controlled-release carbamazepine. The units of carbamazepine can also be made up of mini-tablets or mixture of granules and mini-tablets.

We claim:

1. A process for preparing an oral pharmaceutical composition comprising carbamazepine, wherein the process comprises the steps of:
(i) blending carbamazepine with one or more pharmaceutically acceptable excipients to form a uniform mixture,
(ii) granulating the mixture with one or more organic solvents,
(iii) drying the granulate to obtain discrete units, and
(iv) processing the units into an oral pharmaceutical composition.

2. The process according to claim 1, wherein the organic solvent used to prepare granulating dispersion comprises of methanol, ethanol, propanol, butanol, isopropyl alcohol, acetone, ethylmethyl ketone, dichloro methane trichloro methane and mixtures thereof.

3. The process according to claim 1, wherein the units are formulated plurality of discrete or aggregated units of particles, spheroids, seeds, pellets, beads, granules, mini-tablets, tablets or mixtures thereof.

4. The process according to claim 3, wherein the units are further formulated into compositions of immediate release, controlled-release, or delayed-release.

5. The process according to claim 4, wherein the controlled-release units are prepared by providing a coating of controlled-release polymer over the immediate release units or by blending carbamazepine with controlled-release polymers and other pharmaceutically acceptable excipients to form matrix-type carbamazepine units.

6. The process according to claim 4, wherein the delayed release units are prepared by providing a coating of enteric polymers over carbamazepine containing immediate release or controlled-release units.

7. The process according to claim 4 or 5, wherein the controlled-release units comprise controlled-release polymers selected from cellulose derivatives, starch, gums, alginates, polyvinyl pyrrolidone, acrylic acid derivatives, polyethylene oxides and mixtures thereof.

8. The process according to claim 4 and 6, wherein the delayed release units comprise enteric polymers selected from cellulose acetate phthalate, cellulose acetate trimellitate, hydroxypropyl methylcellulose acetate phthalate, polyvinyl acetate phthalate, hydroxypropyl methylcellulose phthalate, hydroxypropyl methylcellulose acetate succinate, methacrylic acid copolymers and mixtures thereof.

9. The process according to claim 4, wherein the composition further comprises one or more surfactants and pH-modifiers.

10. The process according to claim 9, wherein the surfactant comprises of anionic, cationic, zwitterionic, nonionic surfactants or mixtures thereof.

11. The process according to claim 9, wherein the pH-modifier comprises of citric acid, sodium bicarbonate, monosodium citrate, trisodium citrate, tribasic sodium phosphate, sodium chloride and mixtures thereof.

12. The process according to claim 4, wherein the composition comprises mixture of controlled-release and delayed release units in a ratio of about 20:80 to about 80:20 by weight, to provide a constant release profile up to about 24 hours time period.

13. The process according to claim 4 and 12, wherein the units are filled into capsule or compressed into tablets.

14. The process according to claim 1, wherein the pharmaceutically acceptable excipients comprise one or more of diluents, binders, disintegrants, lubricants, glidants, coloring agents and flavoring agents.

15. A method of treating convulsions, trigeminal neuralgia or bipolar disorder comprising the administration of a pharmaceutical composition prepared by the process of claim 1.
